(19) United States
(12) Patent Application Publication
(10) Pub. No.: US 2025/0228698 A1
(43) Pub. Date: Jul. 17, 2025

(54) ADAPTERS FOR FLUID COLLECTION DEVICES

(71) Applicant: PUREWICK CORPORATION, Covington, GA (US)

(72) Inventors: Tracey E. Knapp, Snellville, GA (US); Robert Hine, Covington, GA (US)

(21) Appl. No.: 18/982,930

(22) Filed: Dec. 16, 2024

Related U.S. Application Data
(63) Continuation of application No. 17/450,864, filed on Oct. 14, 2021, now Pat. No. 12,208,031.
(60) Provisional application No. 63/094,464, filed on Oct. 21, 2020.

(51) Int. Cl. A61F 5/455; A61F 5/44; A61F 5/453; A61M 1/00

(52) U.S. Cl. CPC A61F 5/4556; A61F 5/4404; A61F 5/453; A61M 1/60; A61M 2210/1092; A61M 2210/1096

(57) ABSTRACT

Examples relate to fluid collection assemblies, systems, and methods for collecting fluid from a wearer of the fluid collection assembly. The fluid collection assemblies include a fluid collection device attachable to an adapter. The fluid collection device includes porous material disposed within a fluid impermeable barrier. The adapter includes an interfacing body having at least one orifice and at least one attachment feature for attaching the adapter to the fluid collection device such that the porous material is fluidly connected to the at least one orifice. A fluid collection system includes a fluid collection device operably coupled to a fluid storage container and vacuum source via one or more sections of conduit.

### CROSS-REFERENCE TO RELATED APPLICATIONS

This application is a continuation of U.S. patent application Ser. No. 17/450,864 filed on 14 Oct. 2021, which claims priority to U.S. Provisional Patent Application No. 63/094,464 filed on 21 Oct. 2020, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to devices, systems, and methods of using fluid collection devices. In an embodiment, a fluid collection assembly is disclosed. The fluid collection assembly includes a fluid collection device including a fluid impermeable barrier having an outer surface and an inner surface, the inner surface at least partially defining a chamber, the fluid impermeable barrier also defining an opening extending therethrough, the opening configured to be positioned adjacent to a urethra. The fluid collection device includes a porous material at least partially disposed in the chamber. The fluid collection assembly includes an adapter attachable to the fluid collection device. The adapter includes an interfacing body longitudinally extending along the fluid impermeable barrier, the interfacing body having at least one orifice extending longitudinally along a central region of the interfacing body and tabs extending laterally away from the at least one orifice. The adapter includes at least one attachment feature configured to at least temporarily attach the interfacing body to fluid impermeable barrier with the opening being fluidly connected to the at least one orifice, wherein the at least one attachment feature is operably coupled to the interfacing body.

In an embodiment, a fluid collection system is disclosed. The fluid collection system includes a fluid storage container configured to hold a fluid. The fluid collection system includes a fluid collection device having a fluid impermeable barrier having an outer surface and an inner surface, the inner surface at least partially defining a chamber, the fluid impermeable barrier also defining an opening extending therethrough, the opening configured to be positioned adjacent to a urethra. The fluid collection device includes a porous material at least partially disposed in the chamber. The fluid collection system includes an adapter attached to the fluid collection device, the adapter including an interfacing body longitudinally extending along the fluid impermeable barrier, the interfacing body having at least one orifice extending longitudinally along a central region of the interfacing body and tabs extending laterally away from the at least one orifice. The adapter includes at least one attachment feature configured to at least temporarily attach the interfacing body to fluid impermeable barrier with the opening being fluidly connected to the at least one orifice, wherein the at least one attachment feature is operably coupled to the interfacing body. The fluid collection system includes a conduit including an inlet and an outlet, the outlet being fluidly coupled to the fluid storage container and the inlet being positioned in the chamber. The fluid collection system includes a vacuum source fluidly coupled to one or more of the fluid storage container or the fluid collection device via the conduit, the vacuum source configured to draw fluid from the fluid collection device via the conduit.

In an embodiment, a method to collect fluid is disclosed. The method includes disposing at least one orifice of an adapter of a fluid collection assembly over a urethra of a wearer, the at least one orifice being fluidly connected to a porous material of a fluid collection device of the fluid collection assembly. The method includes receiving fluid from the urethra into the fluid collection device. The method includes removing the fluid from the fluid collection device via a conduit fluidly connected thereto.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is an exploded isometric view of a fluid collection assembly, according to an embodiment.

FIG. 2 is a cross-sectional view of the fluid collection device of the fluid collection assembly of FIG. 1 along the plane A-A, according to at least some embodiments.

FIG. 3 is an isometric view of the adapter of the fluid collection assembly of FIG. 1, according to at least some embodiments.

FIG. 4 an isometric view of the assembled fluid collection assembly of FIG. 1, according to at least some embodiments.

FIG. 5 is an isometric view of an adapter, according to an embodiment.

FIG. 6 is an exploded isometric view of a fluid collection assembly, according to an embodiment.

FIG. 7 is a block diagram of a system for fluid collection, according to an embodiment.

FIG. 8 is a flow diagram of a method to collect fluid, according to an embodiment.

### DETAILED DESCRIPTION

Embodiments disclosed herein are related to assemblies, systems, and methods for collecting fluids from an individual. The fluid collection assemblies, systems, and methods disclosed herein include a fluid collection device and an adapter sized and shaped to allow a fluid collection device for females with average anatomical features to be used to collect urine from wearers whose anatomy differs from an average female wearer. For example, a fluid collection device that is sized and shaped to be used by a female with average anatomical features in the vaginal region may be utilized by females with abnormal anatomical features in the vaginal region and by males with buried penis or micropenis by using the adapter.

The fluid collection devices herein include a fluid impermeable barrier that at least partially defines a chamber. The fluid impermeable barrier also defines an opening extending therethrough that is sized, shaped, and located to be positioned adjacent to a urethra. The fluid collection devices includes a porous material at least partially disposed within the chamber to receive urine from the urethra of a wearer. The fluid(s) collected by the fluid collection devices may include at least one of urine, vaginal discharge, blood, sweat, or other bodily fluids. The fluid collection devices may also include a conduit disposed in the chamber.

The adapter includes an interfacing body and at least one attachment feature connected to the interfacing body. The interfacing body interfaces with the wearer and the at least one attachment feature is sized and shaped to attach the fluid collection device to the interfacing body. The interfacing body longitudinally extends along the fluid impermeable barrier, has at least one orifice extending longitudinally along a central region of the interfacing body, and includes tabs extending laterally away from the at least one orifice. The at least one attachment feature is sized, shaped, and located on the interfacing body to at least temporarily attach the interfacing body to the fluid collection device such that the opening therein is fluidly connected to the at least one orifice. Accordingly, fluids (e.g., urine) collected by the interfacing body, in the at least one orifice, are directed to the porous material in the fluid collection device attached to the interfacing body by the at least one attachment feature.

By providing the adapter, the fluid collection device is retained in position on the wearer with the porous material disposed near the urethra of the wearer. The adapter allows the fluid collection device that is designed to be used for females to be used by males, such as a male with a buried penis or a micropenis. For example, the interfacing body of the adapter interfaces with a male wearer's skin around the penis to retain the adapter in position while the at least one orifice in the adapter that allows the fluid(s) to pass through the adapter to the porous material in the fluid collection device attached to the adapter. Likewise, the adapter allows the fluid collection device to be used on females with anatomical features that are outside of the norm or make maintaining the position of the fluid collection device difficult, such as females with a large pannus or enlarge anatomical features due to obesity. Accordingly, the fluid collection assemblies disclosed herein are sized and shaped to collect fluid(s) from individuals with anatomical features that deviate from the anatomical features of an average female, while preventing leakage, and providing a secure fit on the wearer.

The fluid collection devices and assemblies disclosed herein are configured to be used in fluid collection systems to collect and remove fluids from the wearer of the fluid collection device.

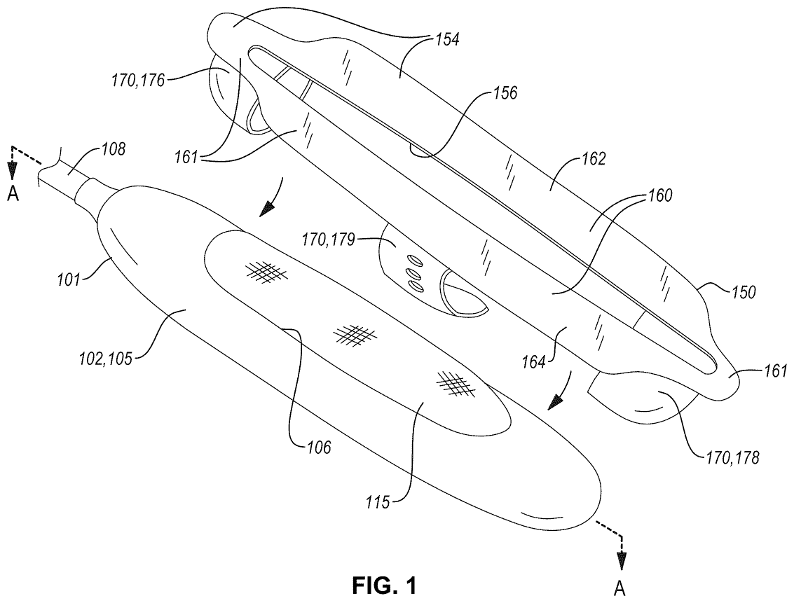

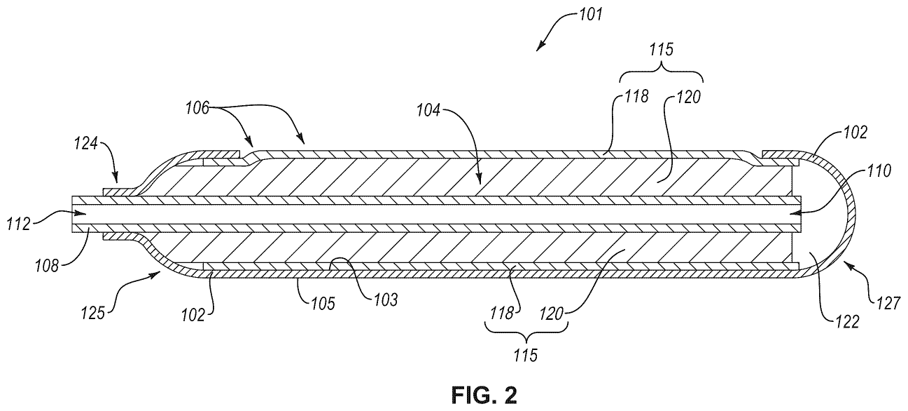

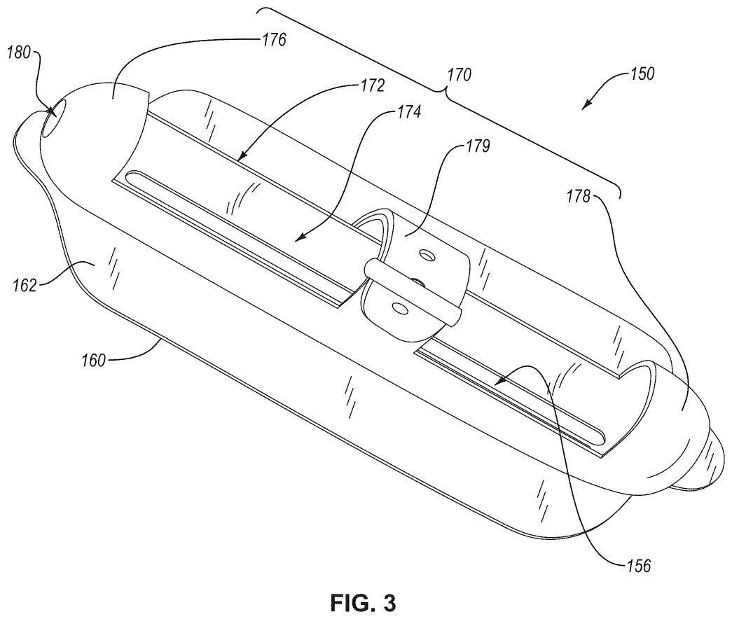

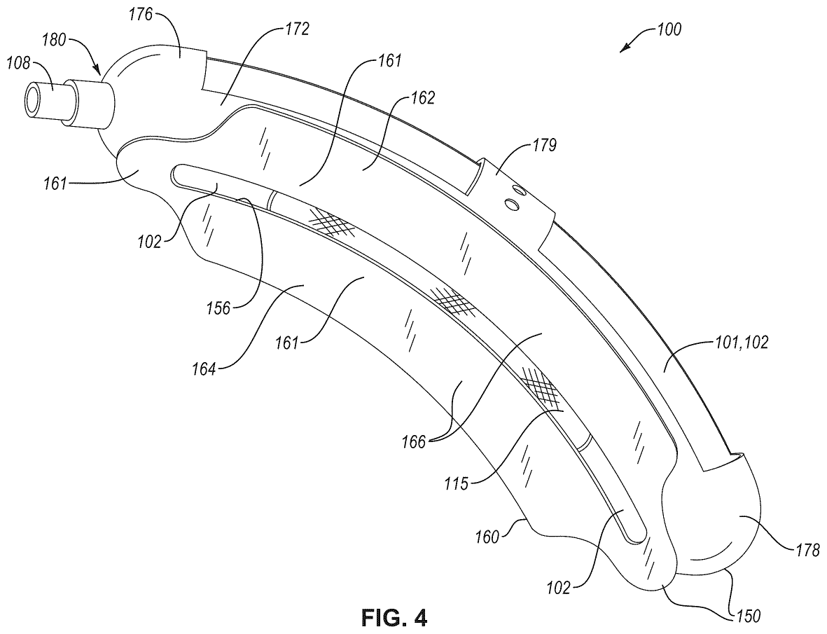

FIG. 1 is an exploded isometric view of a fluid collection assembly 100, according to an embodiment. FIG. 2 is a cross-sectional view of the fluid collection device of fluid collection assembly of FIG. 1 along the plane A-A, according to at least some embodiments. FIG. 3 is an isometric view of the adapter of the fluid collection assembly of FIG. 1, according to at least some embodiments. FIG. 4 an isometric view of the fluid collection assembly 100, according to at least some embodiments. The fluid collection assembly 100 includes a fluid collection device 101 and an adapter 150 for attaching to the fluid collection device 101. For example, the fluid collection device 101 at least partially fits within one or more portions of the adapter 150. Accordingly, the adapter 150 is sized and shaped to retain the fluid collection device 101 at least partially therein.

The fluid collection device 101 is an example of a female fluid collection device for receiving and collecting fluid(s) from a female. The fluid collection device 101 includes a fluid impermeable barrier 102 defining a chamber 104 therein, porous material 115 at least partially disposed in the chamber 104, and an optional conduit 108 at least partially disposed within the chamber 104. The fluid impermeable barrier 102 at least partially defines the chamber 104 (e.g., interior region) and an opening 106. For example, the interior surface(s) 103 of the fluid impermeable barrier 102 at least partially defines the chamber 104 within the fluid collection device 101. The fluid impermeable barrier 102 at least temporarily retains the fluid(s) in the chamber 104. The fluid impermeable barrier 102 may be formed of any suitable fluid impermeable material(s), such as a fluid impermeable polymer (e.g., silicone, polypropylene, polyethylene, polyethylene terephthalate, thermoplastic elastomer(s), a polycarbonate, etc.), a metal film, natural rubber, another suitable material, or combinations thereof. As such, the fluid impermeable barrier 102 substantially prevents the fluid(s) from passing therethrough.

In an example, the fluid impermeable barrier 102 may be air permeable and liquid impermeable. In such an example, the fluid impermeable barrier 102 may be formed of a hydrophobic material that defines a plurality of pores that are air permeable but not liquid permeable. In an example, at least one or more portions of at least an outer surface of the fluid impermeable barrier 102 may be formed from a soft and/or smooth material, thereby reducing chaffing.

In some examples, the fluid impermeable barrier 102 may be tubular (ignoring the opening), such as substantially cylindrical (as shown), oblong, prismatic, or flattened tubes. The fluid impermeable barrier 102 may be sized to fit between the legs of a wearer. During use, an outer surface 105 of the fluid impermeable barrier 102 may at least partially contact the wearer.

The opening 106 provides an ingress route for fluids to enter the chamber 104. The opening 106 may be defined by the fluid impermeable barrier 102, such as by an inner edge of the fluid impermeable barrier 102. For example, the opening 106 is formed in and extends through the fluid impermeable barrier 102, from the outer surface 105 to the inner surface 103, thereby enabling fluid(s) to enter the chamber 104 from outside of the fluid collection device 101. The opening 106 may be located and shaped to be positioned adjacent to a wearer's urethra while the device is in use. At least a portion of porous material(s) disposed in the chamber 104 may be exposed through the opening 106 to allow fluids to move inwardly into the chamber 104, such as via one or more of permeation, suction, or wicking.

The fluid collection device 101 may be positioned proximate to the urethra and urine may enter the chamber 104 via the opening 106. When in use, the opening 106 may be an elongated shape that extends from a first location below the urethra to a second location above the urethra (e.g., at or near the top of the vaginal opening or the pubic region).

The opening 106 may exhibit an elongated shape because the space between the legs of a wearer is relatively narrow when the legs of the wearer are closed, thereby only permitting the flow of the fluid(s) along a path that corresponds to the elongated shape of the opening 106 (e.g., longitudinally extending opening). The opening 106 in the fluid impermeable barrier 102 may exhibit a length that is measured along the longitudinal axis of the fluid collection device 101 that may be at least about 10% of the length of the fluid collection device 101, such as about 25% to about 50%, about 40% to about 60%, about 50% to about 75%, about 65% to about 85%, or about 75% to about 95% of the length of the fluid collection device 101.

The opening 106 in the fluid impermeable barrier 102 may exhibit a width that is measured transverse to the longitudinal axis of the fluid collection device 101 and may be at least about 10% of the circumference of the fluid collection device 101, such as about 25% to about 50%, about 40% to about 60%, about 50% to about 75%, about 65% to about 85%, or about 75% to about 100% of the circumference of the fluid collection device 101. The opening 106 may exhibit a width that is greater than 50% of the circumference of the fluid collection device 101 since the vacuum (e.g., suction) through the conduit 108 pulls the fluid through the porous material 115 and into the conduit 108. The opening 106 may be longitudinally oriented (e.g., having a major axis parallel to the longitudinal axis of the device 101). In some examples (not shown), the opening 106 may be laterally oriented (e.g., having a major axis perpendicular to the longitudinal axis of the device 101).

The fluid collection device 101 includes the porous material 115 disposed in the chamber 104. The porous material 115 may extend across at least a portion (e.g., all) of the opening 106. At least a portion of the porous material 115 may be exposed to an environment outside of the chamber 104 through the opening 106. The porous material 115 may be configured to wick any fluid away from the opening 106, thereby preventing the fluid from escaping the chamber 104. The permeable properties of the porous material referred to herein may be wicking, capillary action, diffusion, or other similar properties or processes, and are referred to herein as “permeable” and/or “wicking.” Such “wicking” may not include absorption of fluid into the porous material. Put another way, substantially no absorption of fluid into the porous material may take place after the material is exposed to the fluid and removed from the fluid for a time. While no absorption is desired, the term “substantially no absorption” may allow for nominal amounts of absorption of fluid into the porous material (e.g., absorbency), such as less than about 10 wt % of the dry weight of the porous material, less than about 7 wt %, less than about 5 wt %, less than about 3 wt %, less than about 2 wt %, less than about 1 wt %, or less than about 0.5 wt % of the dry weight of the porous material. The porous material 115 may also wick the fluid generally towards an interior of the chamber 104.

The porous material 115 may include one or more of a fluid permeable membrane 118 or a fluid permeable support 120. The fluid permeable membrane 118 may include any porous material or a material that may wick the fluid. For example, the fluid permeable membrane 118 may include fabric, such as a gauze (e.g., a silk, linen, or cotton gauze), another soft fabric, or another smooth fabric. The fluid permeable membrane 118 may include spun plastic fibers (e.g., nylon), such as a spun plastic mat or bed. Forming the fluid permeable membrane 118 from gauze, soft fabric, and/or smooth fabric may reduce chaffing caused by the fluid collection device 101.

The fluid collection device 101 may include the fluid permeable membrane 118 disposed in the chamber 104. The fluid permeable membrane 118 may cover at least a portion (e.g., all) of the opening 106. The fluid permeable membrane 118 may be composed to wick any fluid inwardly away from the opening 106, thereby preventing the fluid from escaping the chamber 104.

The porous material 115 of the fluid collection device 101 may include the fluid permeable support 120 disposed in the chamber 104. The fluid permeable support 120 is composed to support the fluid permeable membrane 118 since the fluid permeable membrane 118 may be formed from a foldable, flimsy, or otherwise easily deformable material. For example, the fluid permeable support 120 may be positioned such that the fluid permeable membrane 118 is disposed between the fluid permeable support 120 and the fluid impermeable barrier 102. As such, the fluid permeable support 120 may support and maintain the position of the fluid permeable membrane 118. The fluid permeable support 120 may include any material that may wick the fluid, such as any of the fluid permeable membrane materials disclosed herein. For example, the fluid permeable support 120 may be formed from any fluid permeable material that is less deformable than the fluid permeable membrane 118, such as any of the materials disclosed herein for the fluid permeable membrane 118, in a more dense or rigid form. In some examples, the fluid permeable support 120 may include a porous polymer (e.g., nylon, polyester, polyurethane, polyethylene, polypropylene, etc.) structure or an open cell foam or spun plastic fibers (e.g., nylon fibers). In some examples, the fluid permeable membrane 118 may include gauze and the fluid permeable support may include spun nylon fibers. In some examples, the fluid permeable support 120 may be formed from fabric, felt, gauze, or combinations thereof. In some examples, the fluid permeable support 120 may be formed from a natural material, such as cotton, wool, silk, or combinations thereof. In such examples, the material may have a coating to prevent or limit absorption of fluid into the material, such as a water repellent coating. In some examples, the fluid permeable support 120 may be omitted from the fluid collection device 101. In some examples, the fluid permeable membrane 118 may be optional. For example, the porous material 115 may include only the fluid permeable support 120.

The fluid permeable support 120 may have a greater permeability or a greater ability to wick fluids than the fluid permeable membrane 118, such as to move the fluid inwardly from the outer surface of the fluid collection device 101. In some examples, the permeability or the wicking ability of the fluid permeable support 120 and the fluid permeable membrane 118 may be substantially the same.

The fluid permeable membrane 118 and the fluid permeable support 120 may at least substantially completely fill the portions of the chamber 104 that are not occupied by the conduit 108. In another example, the fluid permeable membrane 118 and the fluid permeable support 120 may not substantially completely fill the portions of the chamber 104 that are not occupied by the conduit 108. In such an example, the fluid collection device 101 includes a reservoir 122 (FIG. 2) in the chamber 104.

The fluid collection device 101 may include the conduit 108, which extends into the chamber 104. As illustrated in FIG. 2, the conduit 108 may be at least partially disposed in the chamber 104. The conduit 108 (e.g., a tube) includes an inlet 110 at a first end region and an outlet 112 at a second end region positioned downstream from the inlet 110. The conduit 108 may extend into the chamber 104 to any point therein. For example, the conduit 108 may be inserted into the chamber at the first end region 125 of the fluid collection device 101 and extend therethrough to the second end region 127 of the fluid collection device 101. The conduit 108 may extend into the fluid impermeable barrier 102 from the first end region 125 (e.g., proximate to the outlet 112) through to the second end region 127 (e.g., opposite the first end region 125) to a point proximate to the reservoir 122 such that the inlet 110 is in fluid communication with the reservoir 122. In some examples (not shown), the conduit 108 may enter the chamber 104 in the second end region 127 and the inlet 110 of the conduit 108 may be disposed in the second end region 127 (e.g., in the reservoir 122 or flush with fluid impermeable barrier 102). The fluid collected in the reservoir 122 may be removed from the chamber 104 via the conduit 108. In some examples, the inlet 110 may be disposed at the end of the fluid permeable support 120 in the second end region 127, such as flush with the end of the fluid permeable support 120. In some examples, the inlet 110 may be disposed within the fluid permeable support 120 such between first end region 125 and the second end region 127.

The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 108 may include silicon or latex. In some examples, the conduit 108 may include one or more portions that are resilient, such as to by having one or more of a diameter or wall thickness that allows the conduit to be flexible. In some examples, the conduit 108 may be frosted or opaque (e.g., black) to obscure visibility of the fluid(s) therein.

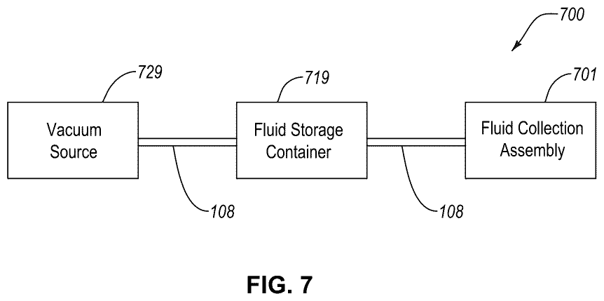

The fluid collection device 101 may be operably coupled to a vacuum source. For example, the conduit 108 fluidly couples an interior region of the chamber 104 with the fluid storage container (FIG. 7) or the vacuum source (FIG. 7). Accordingly, fluids may be removed from the chamber 104 via the conduit 108.

The fluid impermeable barrier 102, the fluid permeable membrane 118 and the fluid permeable support 120 may be sized and shaped to have the conduit 108 at least partially disposed in the chamber 104. For example, at least one of the fluid permeable membrane 118 and the fluid permeable support 120 may be configured to form a space that accommodates the conduit 108. The fluid impermeable barrier 102 may define an aperture 124 sized to receive the conduit 108. The at least one conduit 108 may be disposed in the chamber 104 via the aperture 124. The aperture 124 may be sized and shaped to form an at least substantially fluid tight seal against the conduit 108 thereby substantially preventing the fluid(s) from escaping the chamber 104. The fluid collected in the fluid collection device 101 may be removed from the chamber 104 via the conduit 108.

The porous material 115 (e.g., fluid permeable membrane 118 and the fluid permeable support 120) may not substantially completely fill the portions of the chamber 104 that are not occupied by the conduit 108. The fluid collection device 101 may include the reservoir 122 therein. As shown, the reservoir 122 is a substantially unoccupied portion of the chamber 104. The reservoir 122 may be defined between the fluid impermeable barrier 102 and the porous material 115 (e.g., one or both of the fluid permeable membrane 118 and the fluid permeable support 120). The fluid(s) emitted by the wearer may be wicked into the chamber 104 by the porous material 115 and may flow through the fluid permeable membrane 118 and/or fluid permeable support 120 to the reservoir 122. The fluid impermeable barrier 102 may retain the fluid(s) in the reservoir 122. The reservoir 122 may be located in a portion of the fluid collection device expected to be positioned in a gravimetrically low point of the fluid collection device when worn by a user. While depicted in the second end region 127, the reservoir 122 may be located in any portion of the chamber 104 such as the first end region 125.

In some examples, the fluid permeable support 120 is spaced from at least a portion of the conduit 108 and the reservoir 122 may be defined between the fluid permeable support 120 and the conduit 108.

As shown in FIG. 2, the end of the conduit 108 may extend beyond the fluid permeable membrane 118 and/or fluid permeable support 120, such as into the reservoir 122. In some examples, the inlet 110 may not extend into the reservoir 122. In such examples, the inlet 110 may be disposed within the porous material 115 (fluid permeable membrane 118 and/or fluid permeable support 120) or at a terminal end thereof. For example, an end of the conduit 108 may be coextensive with or recessed within the fluid permeable membrane 118 and/or fluid permeable support 120.

Locating the inlet 110 at or near a location expected to be the gravimetrically low point of the chamber 104 when worn by a user enables the conduit 108 to receive more of the fluid(s) than if inlet 110 was located elsewhere and reduce the likelihood of pooling (e.g., pooling of the fluid(s) may cause microbe growth and foul odors). For instance, the fluid(s) in the fluid permeable membrane 118 and the fluid permeable support 120 may flow in any direction due to capillary forces. However, the fluid(s) may exhibit a preference to flow in the direction of gravity, especially when at least a portion of the fluid permeable membrane 118 and/or the fluid permeable support 120 is saturated with the fluid(s). Accordingly, one or more of the inlet 110 or the reservoir 122 may be located in the second end region 127.

Other embodiments of fluid impermeable barriers, fluid permeable membranes, fluid permeable supports, chambers, conduits and their shapes and configurations are disclosed in U.S. patent application Ser. No. 15/612,325 filed on Jun. 2, 2017; U.S. patent application Ser. No. 15/260,103 filed on Sep. 8, 2016; and U.S. Pat. No. 10,226,376 filed on Jun. 1, 2017, the disclosure of each of which is incorporated herein, in its entirety, by this reference.

Referring to FIGS. 1, 3, and 4, the adapter 150 is sized and shaped to attach (e.g., attachable) to the fluid collection device 101, providing a means for maintaining positioning on a wearer with abnormal physiological features, and directing fluids inwardly toward the fluid collection device 101 therein. The adapter 150 includes an interfacing body 160 and at least one attachment feature 170. The interfacing body 160 is sized and shaped to interface with and maintain a position on a wearer, such as against the skin of the wearer. The interfacing body 160 is also constructed to position the opening and porous material of the fluid collection device attached thereto at least adjacent to the urethra of a wearer. The interfacing body 160 extends longitudinally along the fluid impermeable barrier of the fluid collection device attached thereto. For example, the interfacing body 160 includes a central region 161 at least partially defined by a substantially flat interfacing surface 166 of the interfacing body 160. The central region 161 includes at least one orifice 156 in the interfacing body 160. For example, the at least one orifice 156 may include one or a plurality of orifices extending along the longitudinal axis of the interfacing body 160.

The interfacing body 160 may be formed of a fluid impermeable material, such as one or more of a rubber, a polymer, silicone, or the like. For example, the interfacing body 160 may be made of a low durometer (e.g., 5-40) thermoplastic elastomer (“TPE”). TPE's may include one or more of thermoplastic polyamides (“TPA”), copolyesters (“TPC”), styrenic block copolymers (“TPS”), polyurethanes (“TPU”), polyelefins (“TPO”), or the like. The interfacing body 160 may be relatively thin (e.g., 2 mm to 500 mm, 5 mm to 100 mm, or less than 250 mm) and flexible. The interfacing body 160 may be resiliently flexible.

The central region 161 includes the portion of the interfacing body 160 immediately around the at least one orifice 156 along a longitudinal axis of the interfacing body 160. As shown in FIGS. 1 and 4, the central region 161 may include the longitudinal ends of the interfacing body 160 and the portions of the interfacing body 160 immediately surrounding the at least one orifice 156. As shown, the central region 161 may be longer (e.g., longitudinal length) than the tabs 162 and 164. In some examples, the central region 161 may be the same length as the tabs 162 and 164. In some examples, the central region 161 may be shorter than the tabs 162 and 164. The at least one orifice 156 may be in fluid communication with the porous material in the opening of the fluid collection device attached thereto. For example, the porous material of the fluid collection device is exposed through the at least one orifice 156 of the adapter 150 attached thereto. Accordingly, fluid may be collected in the fluid collection device 101 through the at least one orifice 156 of the adapter 150.

The interfacing body 160 includes tabs 162 and 164 extending laterally away from a longitudinal axis of the adapter 150 or at least one orifice 156 outward. The tabs 162 and 164 may extend at least about 1 cm away from the at least one orifice 156 (e.g., central region 161), such as about 1 cm to about 10 cm, about 1 cm to about 3 cm, about 2 cm to about to 5 cm, about 3 cm to about 6 cm, about 6 cm to about 10 cm, less than 10 cm, or less than 6 cm from the at least one orifice 156 (e.g., central region 161). The tabs 162 and 164 include and at least partially define the substantially flat interfacing surface 166. For example, the interfacing surface 166 may include the central region 161 and the tabs 162 and 164, such as forming a substantially continuous surface on a wearer-facing side of the interfacing body 160. The interfacing surface 166 provides a relatively large amount of surface area (compared to the wearer-facing surfaces of fluid collection device) to retain the adapter 150 in position on a wearer, such as via friction on the wearer's skin or clothes. The interfacing surface may be substantially smooth or may be textured.

Referring to FIGS. 1, 3, and 4, the at least one attachment feature 170 is attached to the interfacing body 160. Accordingly, the at least one attachment feature 170 at least temporarily attaches the interfacing body 160 to the fluid collection device 101. For example, at least one attachment feature 170 is sized and shaped to attach or hold fluid impermeable barrier 102 at least partially therein (e.g., attachable to) in an orientation such that the opening 106 and porous material exposed therein are fluidly connected (e.g., at least partially centered therein or co-extensive therewith) to the at least one orifice 156. The at least one attachment feature 170 may be integrally formed with the interfacing body 160 or may be formed separately and attached to the interfacing body 160 (e.g., adhered, welded, fastened, etc.).

The at least one attachment feature 170 may include at least one strap 179, such as a band, tie, belt, or the like. The at least one strap 179 may span from a first side of the at least one orifice 156 to a second side of the at least one orifice 156 on the side of the interfacing body 160 that faces away from the wearer. Accordingly and as shown in FIG. 4, the fluid collection device 101 may be positioned by the at least one strap 179 to align the opening 106 with the at least one orifice 156. The at least one strap 179 may be located in a longitudinally central portion of the adapter 150. The at least one strap 179 may be located off-center from the longitudinally central portion of the adapter. The at least one strap 179 may include a first portion on a first side of the at least one orifice 156 and a second portion on the second side of the at least one orifice 156, each positioned to meet the other when biased toward each other. The at least one strap 179 may include one or more of a buckle and corresponding belt holes, a clasp, a clamp, hooks, buttons, snap connectors, hook and loop fasteners, adhesive(s), magnets, suction cup(s), or any other retaining system for connecting different portions of the at least one strap 179 to each other. For example and as shown in FIG. 3, the at least one strap 179 may include a first portion on a first side of the at least one orifice 156 and a second portion on the second side of the at least one orifice 156, the first portion having holes for a prong of a buckle or studs and the second portion may have a buckle, loop, or other corresponding feature for attaching the first portion to the second portion.

In some examples, the at least one strap 179 includes a strap on a first side of the at least one orifice 156 which threads through a loop (e.g., eyelet) on the opposite side of the at least one orifice 156. In such examples, the at least one strap 179 may include an attachment feature such as hook and loop fasteners thereon (on a distal and medial portion thereof, respectively) to fasten the at least one strap 179 to itself after threading through the loop. Accordingly, the at least one strap 179 may retain a fluid collection device against the interfacing body 160 with the wicking material fluidly connected to the at least one orifice 156.

In some examples, the at least one strap 179 may include a plurality of straps longitudinally spaced along the adapter 150. For example, two, three, four, or more straps may be spaced longitudinally along the interfacing body 160.

Referring again to FIG. 3, the at least one attachment feature may include an at least partial enclosure 172 defining a cavity 174 sized and shaped to receive the at least a portion of the fluid collection device therein. For example, the at least one attachment feature 170 may include the at least partial enclosure 172 defining the cavity 174 for receiving at least a portion of the fluid impermeable barrier having the porous material at least partially disposed therein. The at least partial enclosure 172 may extend longitudinally along at least a portion of the at least one orifice 156, such as along the entire length of the at least one orifice 156. The at least partial enclosure 172 may include sidewalls that have a size and shape complementary to the outer surfaces of the fluid collection device (e.g., fluid impermeable barrier and porous material). The sidewalls may have a height sufficient to enclose at least a quarter of the circumference of the fluid collection device, such as at least half of the circumference of the fluid collection device, or less than three quarters of the circumference of the fluid collection device. The bottom (or top, depending upon orientation) of the at least partial enclosure 172 includes the at last one orifice 156.

The at least partial enclosure 172 may include one or more endcaps 176 and 178. The first endcap 176 may be sized and shaped to accommodate and retain the first end region 125 of the fluid collection device 101, including the at least one conduit 108 and the aperture 124 (FIG. 2). For example and as shown in FIGS. 3 and 4, the first endcap 176 may have a slot 180 (e.g., gap, hole, space, or cut-out) therein sized and shaped to allow the aperture 124 and/or at least one conduit 108 to pass therethrough. The second endcap 178 may be sized and shaped to accommodate the second end region 127 of the fluid collection device 101. The first and second endcaps 176 and 178 may be attached to or integrally formed with the sidewalls to form the at least partial enclosure 172.

In some embodiments, the first and second endcaps 176 and 178 may be spaced from each other a distance that is equal to or less than the length of the fluid collection device 101. Such spacing places the fluid collection device 101 under compression when positioned within the at least partial enclosure between the endcaps. By placing the fluid collection device 101 in compression between the endcaps, the endcaps may retain the fluid collection device therein. In some examples, the at least partial enclosure may not include the sidewalls. As shown in FIG. 4, by placing the fluid collection device 101 in compression between the endcaps 176 and 178, the adapter may be bent or otherwise manipulated without having the fluid collection device 101 dislodged therefrom, whether used in combination with the at least one strap 179 or not.

One or more of the at least partial enclosure 172, the endcaps 176 and 178, or the at least one strap may be made of a material that is the same as or different from the material of the interfacing body 160. Any components of the adapter 150 (e.g., at least one attachment feature 170) may be formed of a fluid impermeable material, such as one or more of a rubber, a polymer, silicone, or the like. For example, components of the attachment feature 170 may be formed from a low durometer (e.g., 5-40) TPE, such as any of those disclosed herein. The components of the at least one attachment feature 170 may be relatively thin (e.g., 2 mm to 500 mm, 5 mm to 100 mm, or less than 250 mm) and flexible. In some examples, one or more of the components of the attachment feature 170 may be formed from a fabric, such as the at least one strap 179. In some examples, the interfacing body 160 and the at least one attachment feature 170 may be formed of an identical material such as via integral formation (e.g., molding).

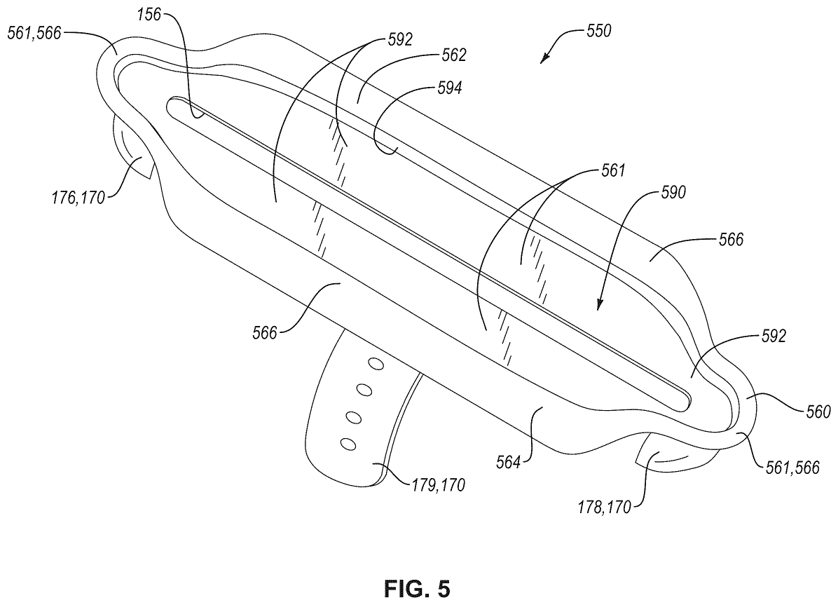

In some examples, the interfacing body may include a sump therein, such as to accommodate anatomical features of a wearer (e.g., a penis or bulbous labia) or direct fluids to the at least one orifice. FIG. 5 is an isometric view of an adapter 550 according to an embodiment. At least some portions of the adapter 550 may be similar or identical to one or more portions of the adapter 150, in one or more aspects. For example, the adapter 550 includes an interfacing body 560 and at least one attachment feature 170 (as disclosed herein). The interfacing body 560 includes a sump 590 on a wearer facing side thereof. The interfacing body 560 includes a central region 561 having the at least one orifice 156 therein and tabs 562 and 564 extending laterally away therefrom. The tabs 562 and 564 in combination with at least the longitudinal ends of the central region 561 may define the interfacing surface 566. The interfacing surface 566 may be substantially continuous around a peripheral edge of the wearer facing surface of the interfacing body 560. The interfacing surface 566 may be substantially flat to provide a seal against the wearer.

The interfacing surface 566 may be spaced from a sump surface 592. For example, the interfacing surface 566 may be spaced above the sump surface 592 by walls 594. The walls 594 may be formed by thicker portions of the interfacing body 560 (e.g., around at least the periphery of the interfacing body 560, such as at the longitudinal ends of the central region 561 and the tabs 562 and 564) than the portions of the interfacing body 560 in the sump 590 at the sump surface 592. Accordingly, the interfacing surface 566 is offset (vertically) from the sump surface 592 to form the sump 590. The at least one orifice 156 is located in and formed by the sump surface 592, such as in the central region 561. In such examples, the sump 590 may provide a space in close proximity with the at least one orifice 156 for anatomy (e.g., penis, fatty tissue, labia, etc.) of a wearer to fit or fluids to collect.

The adapter 550 includes the at least one attachment feature 170 affixed to the interfacing body 560. For example, the at least one attachment feature may include one or more of at least a partial enclosure, the endcaps 176 and 178, or the at least one strap 179. The adapter 550 or the portions thereof

The adapter 550, or the components thereof, may be formed of any of the materials disclosed herein for the adapter 150, or the components thereof. For example, the interfacing body 560 may be formed of rubber, a polymer, silicone, or the like. Despite the thicker portions of the interfacing body 560 at the interfacing surface 566, the interfacing body 560, and the adapter 550 as a whole, may be flexible (e.g., resiliently flexible) to conform to the anatomical features of the wearer when in use.

The fluid collection device 101 may be affixed to the adapter 550 to form a fluid collection assembly, as disclosed herein with respect to fluid collection assembly 100.

In some examples, the at least one orifice may be larger than is depicted in FIGS. 1-5. In some examples, the fluid collection device may be input into or onto the adapter via the wearer-facing side of the adapter, such as through the at least one orifice.

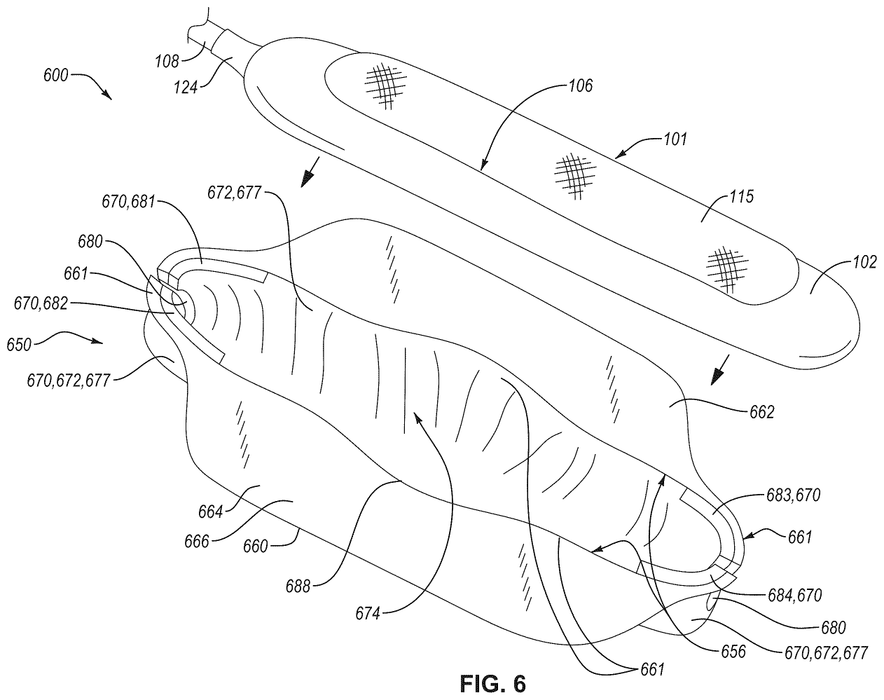

FIG. 6 is an exploded isometric view of a fluid collection assembly 600, according to an embodiment. The fluid collection assembly 600 includes the fluid collection device 101 and the adapter 650. The fluid collection device 101 may be positioned at least partially within the adapter 650. The adapter 650 includes an interfacing body 660 having an orifice 656 therein. The orifice 656 is sized and shaped to accommodate the fluid collection device 101 at least partially therein, such as having outer dimensions slightly larger than (e.g., 1%-5% larger), equal to, or slightly less than (e.g., 1%-5% smaller) the outer dimensions of the fluid collection device 101. The orifice 656 is located in a central region 661 of the interfacing body 660. The interfacing body includes tabs 662 and 664 extending laterally way from (e.g., perpendicular to) the longitudinal axis of the adapter 650. The central region 661 and the tabs 662 and 664 collectively form the interfacing surface 666. The interfacing surface 666 is substantially flat. The interfacing surface 666 may be smooth or may be textured.

The at least one orifice 656 may be fluidly connected to a cavity 674. The cavity 674 may be part of the at least one attachment feature 670. The at least one attachment feature 670 may be similar or identical to the at least one attachment feature 170, in one or more aspects. The cavity 674 may be formed by at least one wall 677 of an at least partial enclosure 672. For example, the attachment feature 670 may include at least one wall 677. The at least one wall 677 may be sized and shaped to accommodate the fluid collection device 101 therein, such as to provide an interference fit or compression fit with the fluid collection device 101. The at least one wall 677 may include endcaps formed therein. The endcaps may be similar or identical to the endcaps 176 and 168 in one or more aspects. The at least one wall 677 may extend between the endcaps. Accordingly, the cavity 674 formed by the at least partial enclosure 672 and may collect and retain fluids therein.

The endcaps of the at least one wall 677 may include slots 680 therein. The slots 680 may extend from the interfacing surface 666 into the at least one wall 677. At least one of the slots 680 may include a cut-out sized and shaped to accommodate the aperture 124 and/or conduit 108 of the fluid collection device 101, such as at the longitudinal end of the at least one wall 677. At least one of the slots 680 may include a cut or slit from the interfacing surface to a midpoint in the at least one wall 677. In some examples, only one end of the at least partial enclosure 672 may include a slot 680, such as to accommodate the conduit 108 and/or aperture 124. The slots 680 may allow the at least one orifice 656 to be opened wider to allow insertion of the fluid collection device 101 into the cavity 674. For example, the slots 680 may allow the at least partial enclosure 672 to at least partially open up to receive the fluid collection device 101.

The at least one attachment feature 670 may include one or more flaps 681-684 extending over a portion of the cavity 674. The one or more flaps 681-684 may protrude over a portion of the fluid collection device 101 disposed in the cavity 674 to help retain the fluid collection device 101 in the cavity 674. The one or more flaps 681-684 may be located in the first end region and the second end region of the at least one orifice 656. The one or more flaps 681-684 may protrude over the cavity at least about 1 mm, such as about 1 mm to about 1 cm, about 1 mm to about 5 mm, about 5 mm to about 1 cm, or less than about 1 cm. In some examples, additional flaps may be located around the orifice 656, such as in the medial portions thereof between the end regions of the orifice 656. The one more flaps 681-684 may be made of the same material or a similar material as any other portion of the adapter 650, such as the interfacing body 660 or the at least one attachment feature 670. For example, one or more portions of the adapter 650 may be made of any of the materials disclosed herein, such as a rubber, a polymer (e.g., TPE), or silicone. Accordingly, the adapter 650 may be flexible as disclosed herein with respect to the adapters 150 and 550 disclosed herein.

As shown in FIG. 6, the fluid collection device 101 may be positioned into the attachment feature 670 of the adapter 650 via the at least one orifice 656 on the wearer-facing side of the adapter. The opening 106 (and the porous material 115 exposed therethrough) may be at least partially centered in, and in fluid communication with, the at least one orifice 656 facing outward to collect fluids therethrough.

In some examples, a medial portion 688 of the at least one orifice 656 and cavity 674 may be wider than the end regions thereof. In such examples, the wider medial portion 688 may be expected to be positioned around the urethra of a wearer to collect fluid from a larger area than if the medial portion was not wider. The wider medial portion 688 may also aid a user to position the fluid collection device 101 in the cavity 674 with relative ease. In some examples, the medial portion of the cavity 674 and the at least one orifice 656 may not include a wider portion.

The fluid collection assembly 600 may be formed by positioning the fluid collection device 101 within the cavity 674 of the adapter 650. The conduit 108 of the fluid collection device 101 may be positioned through the slot in an end of the at least partial enclosure 672 of the adapter 650.

Any of the fluid collection assemblies, fluid collection devices, and adapters disclosed herein may be utilized in a fluid collection system. FIG. 7 is a block diagram of a system 700 for fluid collection, according to an embodiment. The system 700 includes a fluid collection assembly 701, a fluid storage container 719, and a vacuum source 729. The fluid collection assembly 701, the fluid storage container 719, and the vacuum source 729 may be fluidly coupled to each other via one or more conduits 108. For example, the fluid collection device of the fluid collection assembly 701 may be operably coupled to one or more of the fluid storage container 719 or the (portable) vacuum source 729 via the conduits 108.

The fluid collection assembly 701 may include any of the fluid collection assemblies, fluid collection devices, or adapters disclosed herein. For example, the fluid collection device of the fluid collection assembly 701 may include a fluid impermeable barrier defining an opening and porous material exposed through the opening, as disclosed herein. The fluid collection device of the fluid collection assembly 701 may include the conduit 108 including an inlet and an outlet as disclosed herein. The outlet may be fluidly coupled to the fluid storage container 719 and the inlet may be positioned in the fluid collection device of the fluid collection assembly 701 such as in a portion of the chamber therein selected to be at a gravimetrically low point of the fluid collection device when the fluid collection assembly 701 is worn by a user (e.g., reservoir).

The conduit 108 is coupled to and at least partially extends between one or more of the fluid storage container 719 and the vacuum source 729. In an example, the conduit 108 is directly connected to the vacuum source 729. In such an example, the conduit 108 may extend from the fluid collection device of the fluid collection assembly 701 by at least one foot, at least three feet, at least 5 feet, or at least ten feet. In another example, the conduit 108 may be indirectly connected to at least one of the fluid storage container 719 and the vacuum source 729. In some examples, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet (110, FIG. 2) and the outlet (112, FIG. 2) may fluidly couple (e.g., directly or indirectly) the vacuum source 729 to the chamber (104 or the reservoir 122 therein, FIG. 2) of the fluid collection device, such as via one or more connectors thereon. In an example, the inlet and/or the outlet may form a male connector. In another example, the inlet and/or the outlet may form a female connector. In an example, the inlet and/or the outlet may include ribs that are configured to facilitate secure couplings. In an example, the inlet and/or the outlet may form a tapered shape. In an example, the inlet and/or the outlet may include a rigid or flexible material.

Fluid (e.g., urine or other bodily fluids) collected in the fluid collection assembly 701 may be removed from the fluid collection device therein via the conduit 108. For example, the first open end of the conduit 108 in the fluid collection device is fluidly connected to the second open end of the conduit 108 which may be disposed in the fluid storage container 719, or the vacuum source 729. The suction force may be introduced into the interior region of the fluid collection device via the first open end of the conduit 108 responsive to a suction (e.g., vacuum) force applied at the second end of the conduit 108. The suction force may be applied to the second open end of the conduit 108 by the vacuum source 729 either directly or indirectly. As the vacuum source 729 applies a vacuum/suction in the conduit 108, the fluid(s) in the chamber (e.g., at the second end region such as in the reservoir 122) of the fluid collection device of the fluid collection assembly 701 are drawn into the inlet and out of the fluid collection device via the conduit 108.

The suction force may be applied indirectly via the fluid storage container 719. For example, the second open end (e.g., outlet) of the conduit 108 may be disposed within the fluid storage container 719 and an additional conduit 108 may extend from the fluid storage container 719 to the vacuum source 729. Accordingly, the vacuum source 729 may indirectly apply suction to the fluid collection device via the fluid storage container 719. In such examples, the vacuum source 729 may provide a vacuum/suction through the fluid storage container to the fluid collection device to provide suction in the chamber of the fluid collection device. As the fluid is drained from the chamber of the fluid collection device, the fluid may travel through the first section of conduit 108 to the fluid storage container 719 where it may be retained.

In some examples, the suction force may be applied directly via the vacuum source 729. For example, the second open end of the conduit 108 may be disposed within the vacuum source 729. An additional conduit 108 may extend from the vacuum source 729 to a point outside of the fluid collection device, such as to the fluid storage container 719. In such examples, the vacuum source 729 may be disposed between the fluid collection device and the fluid storage container 719. In examples, the fluid storage container 719 may include a bag (e.g., drainage bag), a bottle or cup (e.g., collection jar), a canister, or any other enclosed container for storing bodily fluids such as urine. In some examples, the fluid storage container 719 may be a rigid container.

The vacuum source 729 may include a wall mounted vacuum line, such as found in hospital rooms. The vacuum source 729 may include one or more of a manual vacuum pump, and electric vacuum pump, a diaphragm pump, a centrifugal pump, a hand pump, a displacement pump, a magnetically driven pump, a peristaltic pump, or any pump configured to produce a vacuum. In examples, the vacuum source 729 may be powered by one or more of a power cord (e.g., connected to a power socket), one or more batteries, or even manual power (e.g., a hand operated vacuum pump). The vacuum sources 729 disclosed herein may include one or more of a switch, a button, a plug, a remote, or any other device suitable to activate the vacuum source 729.

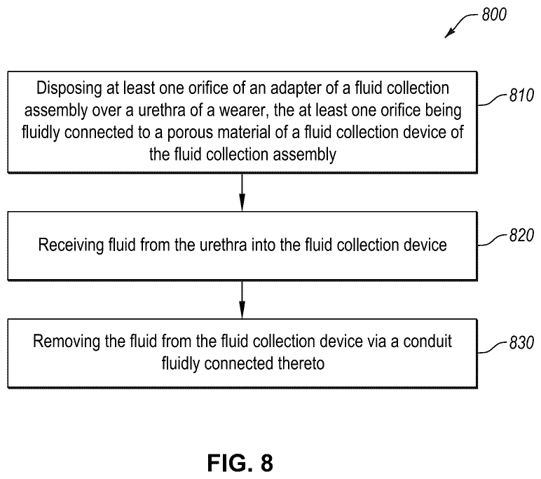

FIG. 8 is a flow diagram of a method to collect fluid, according to an embodiment. The method 800 includes block 810, which recites “disposing at least one orifice of an adapter of a fluid collection assembly over a urethra of a wearer, the at least one orifice being fluidly connected to a porous material of a fluid collection device of the fluid collection assembly.” Block 810 may be followed by Block 820, which recites “receiving fluid from the urethra into the fluid collection device.” Block 820 may be followed by Block 830, which recites “removing the fluid from the fluid collection device via a conduit fluidly connected thereto.”

Blocks 810, 820, 830 of the method 800 are for illustrative purposes. For example, the block 810, 820, 830 of the method 800 may be performed in different orders, split into multiple blocks, modified, supplemented, or combined. In an example, one or more of the blocks 810, 820, 830 of the method 800 may be omitted from the method 800. For example, the block 810 of disposing at least one orifice of an adapter of a fluid collection assembly over a urethra of a wearer, the at least one orifice being fluidly connected to a porous material of a fluid collection device of the fluid collection assembly may be omitted.

Block 810 recites “disposing at least one orifice of an adapter of a fluid collection assembly over a urethra of a wearer, the at least one orifice being fluidly connected to a porous material of a fluid collection device of the fluid collection assembly.” The fluid collection assemblies or components thereof may be similar or identical to any of the fluid collection devices (e.g., 100 or 600) disclosed herein, in one or more aspects. For example, the fluid collection device or components thereof may be similar or identical to any of the fluid collection devices (e.g., 101) disclosed herein, in one or more aspects. The adapter of the fluid collection assembly or components thereof may be similar or identical to any of the adapters (e.g., 150, 550, 650) disclosed herein, in one or more aspects. For example, the fluid collection device may include a fluid impermeable barrier having an outer surface and an inner surface, the inner surface at least partially defining a chamber, the fluid impermeable barrier also defining an opening extending therethrough, the opening configured to be positioned adjacent to a urethra. The fluid collection device may include the porous material at least partially disposed in the chamber. The adapter may include an interfacing body longitudinally extending along the fluid impermeable barrier, the at least one orifice extending longitudinally along a central region of the interfacing body, and tabs of the interfacing body extend laterally away from the at least one orifice. The adapter may include at least one attachment feature sized and shaped to at least temporarily attach the interfacing body to the fluid impermeable barrier with the opening being fluidly connected to the at least one orifice, wherein the at least one attachment feature is operably coupled to the interfacing body. The method 800 may use any of the fluid collection systems disclosed herein.

Disposing at least one orifice of an adapter of a fluid collection assembly over a urethra of a wearer may include disposing the adapter on the wearer, such as against the skin of the wearer. For example, disposing at least one orifice of an adapter of a fluid collection assembly over a urethra of a wearer may include disposing the tabs of the interfacing body in contact with (skin of) the wearer. Disposing at least one orifice of an adapter of a fluid collection assembly over a urethra of a wearer may include disposing the at least one orifice over a female urethra. Disposing at least one orifice of an adapter of a fluid collection assembly over a urethra of a wearer may include positioning a longitudinal axis of the at least one orifice in alignment between the labia of the wearer. For example, disposing at least one orifice of an adapter of a fluid collection assembly over a urethra of a wearer may include disposing the at least one orifice over a male urethra, such as over the urethra of a male having buried penis or a micropenis. Disposing at least one orifice of an adapter of a fluid collection assembly over a urethra of a wearer may include disposing the tabs of the interfacing body under a pannus of the wearer. Disposing at least one orifice of an adapter of a fluid collection assembly over a urethra of a wearer may include disposing the tabs of the interfacing body on one or more of the pubic region, inguinal region, inner thighs, or labia of the wearer.

In some examples, block 810 may include positioning the at least one orifice within 2 cm of the urethra of the wearer, such as 0.1 mm to 2 cm, 0.1 mm to 5 mm, 5 mm to 1 cm, 1 cm to 2 cm, less than 1.5 cm, less than 1 cm, less than 5 mm, more than 0.1 mm, or more than 1 mm away from the urethra of the wearer. Disposing the at least one orifice of an adapter of a fluid collection assembly over a urethra of a wearer may include positioning the at least a portion of the fluid collection assembly between the thighs of the wearer.

The block 820 of receiving fluid from the urethra into the fluid collection device may include receiving the fluid in the porous material. Receiving fluid from the urethra into the fluid collection device may include receiving the fluid in the porous material via the at least one orifice. The block 820 of receiving fluid from the urethra into the fluid collection device may include wicking the fluid(s) from the urethra via wicking the porous material, such as via a fluid permeable membrane and a fluid permeable support. Receiving fluid from the urethra into the fluid collection device may include wicking the fluid into the chamber of the fluid collection device via the porous material. In some examples, the block 820 may include flowing the fluid towards a portion of the chamber of the fluid collection device that is fluidly coupled to an inlet of a conduit (which may be in fluid communication a vacuum source). For instance, block 820 may include flowing the fluid(s) to a substantially unoccupied portion of the chamber (e.g., a reservoir), to a gravimetrically low point of the chamber, etc. In some examples, receiving fluid(s) from the urethra into a chamber of the fluid collection device may include wicking the fluid (e.g., urine) into the chamber via the fluid permeable membrane and fluid permeable support of the fluid collection device via the at least one orifice in the adapter.

The block 830 of removing the fluid from the fluid collection device may include applying suction with a vacuum source fluidly connected to the conduit effective to suction the fluid from the chamber via the conduit. Applying suction may include using any of the vacuum sources disclosed herein. The vacuum source may be spaced from the fluid collection device, such as via one or more portions of conduit. In some examples, a fluid storage container is disposed between the vacuum source and the fluid collection device. The block 830 may include activating the vacuum source (e.g., suction device) in fluid communication with the inlet of the conduit in the fluid collection device. In some examples, activating the vacuum source may include supplying power to the vacuum source by one or more of flipping an on/off switch, pressing a button, plugging the vacuum source into a power outlet, putting batteries into the vacuum source, etc. In some examples, the vacuum source may include a hand operated vacuum pump and applying suction with a vacuum source may include manually operating the hand operated vacuum pump effective to suction the fluid(s) from the chamber via the conduit.

In some examples, applying suction with a vacuum source effective to suction the fluid(s) from the chamber via a conduit disposed therein and fluidly coupled to the vacuum source may be effective to remove at least some fluid (e.g., urine) from the chamber (e.g., interior region) of the fluid collection device. In some examples, applying suction with a vacuum source effective to suction the fluid(s) from the chamber via a conduit disposed therein and fluidly coupled to the vacuum source may be effective to transfer at least some of the fluid from the chamber of the fluid collection device to a fluid storage container (e.g., a bottle or bag). In some examples, applying suction with a vacuum source effective to suction the fluid(s) from the chamber may include removing fluid from one or more of a reservoir, fluid permeable support, or fluid permeable membrane of the fluid collection device.

Removing the fluid from the fluid collection device may include allowing the fluid to drain from the fluid collection device of the fluid collection assembly via gravity.

In an example, the method 800 may include collecting the fluid(s) that are removed from the fluid collection device, such as into the fluid storage container that is spaced from the fluid collection device and fluidly coupled to the conduit. The fluid storage container may include any of the fluid storage containers disclosed herein.

The method 800 may include forming the fluid collection assembly. Forming the fluid collection assembly may include positioning the fluid collection device within the adapter. positioning the fluid collection device within the adapter may include positioning the porous material of the fluid collection device in fluid connection with the at least one orifice, such as aligning the porous material within the at least one orifice. Positioning the fluid collection device within the adapter may include securing the fluid collection device with or within the at least one attachment feature (e.g., strap, at least partial enclosure, end caps, etc.).

The adapter of the fluid collection assemblies, systems, and methods disclosed herein allow female fluid collection devices to be utilized to collect fluids from wearers with anatomical features that deviate from the average female wearer. For example, the adapter of the fluid collection assemblies, systems, and methods disclosed herein provide for secure positioning of female fluid collection devices on wearer's with abnormal female anatomical features in the pelvic region (e.g., large pannus, enlarged anatomical features due to obesity, or the like) or even male wearers who have buried penis, micropenis, or the like.

While various aspects and embodiments have been disclosed herein, other aspects and embodiments are contemplated. The various aspects and embodiment disclosed herein are for purposes of illustration and are not intended to be limiting.

1. (canceled)

2. A fluid collection assembly, comprising:
a fluid collection device, including:
a fluid impermeable barrier having an outer surface and two opposing convex ends, the outer surface exhibiting a generally cylindrical shape, the fluid impermeable barrier at least partially defining a chamber, an opening extending therethrough, and an aperture configured to receive a conduit; and
a porous material at least partially disposed in the chamber; and
an adapter attached to the fluid collection device, the adapter including:
an interfacing body having at least one orifice and a central region, the central region including an interior surface at least partially exhibiting a generally cylindrical shape; and
at least one endcap including a concave interior surface configured to receive and abut at least one of the two opposing convex ends of the fluid impermeable barrier.

3. The fluid collection assembly of claim 2, wherein the fluid impermeable barrier and the porous material define a reservoir therebetween; and
further comprising a conduit partially received in the aperture, an inlet of the conduit is disposed in or adjacent to the reservoir.

4. The fluid collection assembly of claim 2, wherein the fluid impermeable barrier exhibits a longitudinal axis.

5. The fluid collection assembly of claim 4, wherein the interfacing body longitudinally extends along the fluid impermeable barrier.

6. The fluid collection assembly of claim 4, wherein at least a portion of the interfacing body is generally cylindrical and exhibits a longitudinal axis that is generally aligned with the longitudinal axis of the fluid impermeable barrier when the fluid collection device is disposed in the central region.

7. The fluid collection assembly of claim 2, wherein the at least one orifice extends longitudinally along the central region of the interfacing body.

8. The fluid collection assembly of claim 2, wherein the interfacing body includes tabs extending laterally away from the at least one orifice.

9. The fluid collection assembly of claim 8, wherein the tabs include a substantially flat interfacing surface for interfacing with a wearer's skin.

10. The fluid collection assembly of claim 9, wherein the interfacing body includes a sump surface defining the at least one orifice, the substantially flat interfacing surface surrounds and is offset from the sump surface.

11. The fluid collection assembly of claim 8, wherein the interfacing body, excluding the tabs, exhibits a generally cylindrical shape.

12. The fluid collection assembly of claim 2, wherein the adaptor includes an enclosure including an innermost surface defining a cavity, at least substantially all of the innermost surface sized and shaped to directly contact the outer surface of the fluid impermeable barrier when the fluid collection device is received in the cavity.

13. The fluid collection assembly of claim 2, wherein the at least one endcap define a slot extending therethrough.

14. The fluid collection assembly of claim 13, wherein at least a portion of the slot exhibits a size that generally corresponds to a size of at least one of the aperture or the conduit.

15. The fluid collection assembly of claim 13, wherein the slot extends completely through the at least one of the at least one endcap, a maximum dimension of the slot extending generally perpendicularly to the to a longitudinal axis of the fluid impermeable barrier when the fluid impermeable barrier is positioned within the central region, the slot extending generally away from and open to an elongated opening that is configured to receive all of the fluid collection device.

16. The fluid collection assembly of claim 13, further comprising a conduit partially received in the aperture; and
wherein the fluid impermeable barrier includes a longitudinal axis and a portion of the conduit extending through the aperture and the slot is generally parallel to the longitudinal axis.

17. The fluid collection assembly of claim 2, wherein the interfacing body defines an elongated opening that is distinct and separate from the at least one orifice, the elongated opening is configured to receive substantially all of the fluid collection device therethrough.

18. The fluid collection assembly of claim 2, wherein the at least one orifice is configured to receive substantially all of the fluid collection device therethrough.

19. The fluid collection assembly of claim 2, wherein the adapter further includes at least one attachment feature configured to at least temporarily attach the interfacing body to the fluid impermeable barrier.

20. The fluid collection assembly of claim 19, wherein the at least one attachment feature includes at least one strap.

21. A fluid collection system, comprising:
a fluid collection device, including:
a fluid impermeable barrier having an outer surface and two opposing convex ends, the outer surface exhibiting a generally cylindrical shape, the fluid impermeable barrier at least partially defining a chamber, an opening extending therethrough, and an aperture configured to receive a conduit; and
a porous material at least partially disposed in the chamber;
an adapter attached to the fluid collection device, the adapter including:
an interfacing body having at least one orifice and a central region, the central region including an interior surface at least partially exhibiting a generally cylindrical shape; and
at least one endcap, the at least one endcap including a concave interior surface configured to receive and abut at least one of the two opposing convex ends of the fluid impermeable barrier;
a fluid storage container configured to hold a fluid;
the conduit fluidly coupled to the fluid storage container and the chamber, wherein a portion of the conduit extending through the aperture; and
a vacuum source fluidly coupled to one or more of the fluid storage container or the fluid collection device, the vacuum source configured to draw fluid from the fluid collection device via the conduit.
